(19) United States
(12) Patent Application Publication
(10) Pub. No.: US 2021/0030681 A1
(43) Pub. Date: Feb. 4, 2021

(54) MULTIPARTICULATE SOLID DOSAGE FORM HAVING AN ELASTIC TEXTURE

(71) Applicant: DSM IP ASSETS B.V., Heerlen (NL)

(72) Inventor: Jean-Claude TRITSCH, Kaiseraugst (CH)

(21) Appl. No.: 17/046,107

(22) PCT Filed: Apr. 10, 2019
(86) PCT No.: PCT/EP2019/059005

§ 371 Date: Oct. 8, 2020

(30) Foreign Application Priority Data
Apr. 10, 2018 (EP) 18166672.8

(51) Int. Cl. A61K 9/16; A61K 9/20; A61K 31/202; A23L 33/12; A23P 10/30

(52) U.S. Cl. CPC A61K 9/1652; A61K 9/2081; A61K 31/202; A61K 9/5089; A23L 33/12; A23P 10/30; A61K 9/1682

(57) ABSTRACT

The present invention relates to a multiparticulate solid dosage form (1) which has an elastic texture and which contains a plurality of microcapsules (2) having a core (2a) and a shell (2b) that are embedded in an edible matrix (3). Microcapsules (2) contain an active ingredient which may be a pharmaceutical drug and/or a micronutrient. The multiparticulate solid dosage form of the invention is obtainable by a method wherein a mixture comprising water, microcapsules and starch particles is casted. The starch particles swell or dissolve only after casting.

### TECHNICAL FIELD

The present invention relates to the manufacture of multiparticulate solid dosage forms for oral administration.

Multiparticulate solid dosage forms are produced by compressing pellets together with customary excipients and additives to produce tablets. Said pellets contain an active ingredient. Pellets may also have a functional coating to control the release of the active ingredient.

### BACKGROUND OF THE INVENTION

WO 2013/092589 discloses a multiple unit pellet tablet formulation for oral administration. On page 4 of WO 2013/092589, issues relating to the compression of pellets are discussed.

Compressing pellets entails the risk that the functional coating of the pellets will be damaged, resulting in considerable risks for the patient, as described in US 2010/247647, paragraph [0006].

A typical tablet press applies a pressure of approx. 75 kN/cm². So far, there is no solution to prevent damage or to avoid fracturing of the pellets when such high pressures are applied.

Microcapsules are smaller than pellets. However, similar problems occur when compressing microcapsules together with customary excipients and additives to give a multiparticulate solid dosage form for oral consumption.

WO 2009/010305 relates to the reduction of extrusion loss when pressing a formulation of a lipophilic health ingredient to tablets, said lipophilic health ingredient being encapsulated by modified starch.

Thus, there is a need for an improved method for producing multiparticulate solid dosage forms.

### SUMMARY OF THE INVENTION

Microcapsules are defined as small particles of solids, or droplets of liquids, inside a thin coating of a shell material such as beeswax, starch, gelatin, hydrocolloids or polyacrylic acid. They are used, for example, to protect against oxidation and/or to control the rate of release of an active ingredient such as an enzyme, a flavor, a nutrient, a drug, etc.

The problem to be solved by the present invention is avoiding cracks, holes, etc. in the shell of the microcapsules. Such damage typically occurs when a mixture comprising microcapsules and customary excipients is compressed to produce a multiparticulate solid dosage form.

Cracks in the shell of microcapsules may result in leakage of the active ingredient which is encapsulated by said shell. Thus, another problem to be solved by the present invention is avoiding leakage of the active ingredient when producing a multiparticulate solid dosage form.

Some active ingredients have a bad taste or off-flavor. The leakage of such an active ingredient renders the solid dosage form unusable. Thus, another problem to be solved by the present invention is to lower the number of defective products when manufacturing multiparticulate solid dosage forms.

Furthermore, patient compliance with a solid dosage form having an off-flavor is low. Thus, another problem to be solved by the present invention is increasing patient compliance with multiparticulate solid dosage forms which comprise an active ingredient that has an off-flavor or that becomes smelly upon oxidation.

Surprisingly, multiparticulate solid dosage forms can be manufactured by casting a mixture comprising water, microcapsules and starch particles. Typically, said particulate starch swells slowly when they come into contact with water. Because the particulate starch swells slowly, the mixture comprising microcapsules has a low viscosity before and during casting. After casting, however, the viscosity increases massively such that what was previously liquid turns (at the latest after drying) into a solid dosage form having an elastic texture.

Non-particulate starch swells quickly. As a result, a mixture comprising non-particulate starch has a high viscosity right from the beginning. Casting of a high-viscous mixture is not possible as such mixture does not flow sufficiently.

During the manufacturing process of the invention, no tablet press is used and thus, the above-mentioned problems (e.g. cracks or holes in the shell or coating of the pellets, microcapsules etc.) do not occur.

The present invention relates to a method of producing a multiparticulate solid dosage form which has an elastic texture, wherein a mixture comprising water, microcapsules and starch particles is casted, characterized in that said microcapsules encapsulate at least one pharmaceutical drug and/or at least one micronutrient. The preferred micronutrient is docosahexaenoic acid (DHA).

The present invention also relates to a multiparticulate solid dosage form which is obtained by casting, wherein said dosage form comprises at least 100 microcapsules.

### DETAILED DESCRIPTION OF THE INVENTION

The multiparticulate solid dosage form of the present invention is obtained by casting a specified mixture at a temperature of preferably less than 100° C., more preferably less than 90° C. and most preferably at a temperature of less than 85° C. No tablet press is used.

Thus, the multiparticulate solid dosage form of the invention is referred to as a “casted” multiparticulate solid dosage form.

A preferably automated mogul line may be used for casting. So far, mogul lines have only been used for casting confectionery products. A well-known provider of mogul lines is MILTSAM (Miltenberg & Samton, Inc., Wilton, Conn. 06897, USA).

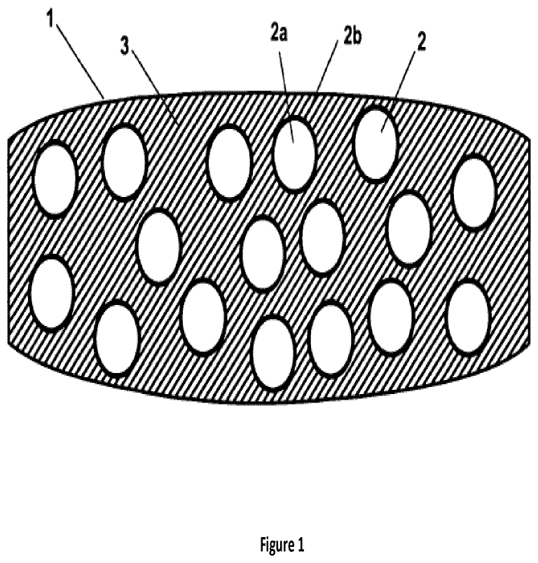

FIG. 1 schematically illustrates a multiparticulate solid dosage form (1) that contains a plurality of microcapsules (2) having a core (2a) and a shell (2b) that are embedded in edible matrix (3). Typically, the edible matrix (3) comprises swelled and/or dissolved starch particles. Microcapsules that encapsulate a pharmaceutical drug and/or a micronutrient comprise a pharmaceutical drug and/or a micronutrient in core (2a). In some embodiments of the invention, core (2a) comprises two or more non-identical drugs and/or two or more non-identical micronutrients.

The multiparticulate solid dosage form of the invention is obtained by casting an aqueous mixture which comprises microcapsules. In the context of the present invention, cells (such as bacterial cells) and viruses are not encompassed by the term “microcapsules”.

In the context of the present invention, the term “solid dosage form” is limited to a dosage form that is administered orally or that can be eaten. This includes small dosage forms that can be swallowed as a whole, similar to a tablet. However, it also includes dosage forms which are too big to be swallowed without chewing. The person skilled in the art understands that the size of the dosage form needs to be adapted to the size and nature of the microcapsules that are embedded in the edible matrix. If microcapsules with a functional coating are used, the dosage form should not be chewed as chewing could damage the functional coating. In such cases, the dosage form must be small enough to be swallowed without chewing. Preferably, solid dosage form of the invention has a weight of at least 1 mg, more preferably of at least 100 mg and most preferably of at least 1 g.

In the context of the present invention, the term “functional coating” refers to a layer that covers shell (2b) of microcapsule (2), such the active ingredient is released in a controlled or sustained manner. The microcapsules according to the invention have preferably no functional coating.

The multiparticulate solid dosage form according to the invention can be manufactured by using a method that is described in EP 2 015 642 B1 and EP 2 410 865 B1. Said patents do not disclose compositions which comprise microcapsules; they relate to confectionery articles. Any of the starch particles disclosed in EP 2 015 642 B1 and EP 2 410 865 B1 can be used in the context of the present invention.

In preferred embodiment of the present invention, a casting method similar to the method described in EP 2 410 865 B1 (cf. paragraphs [0039] to [0045]) is used.

According to the present invention, a mixture comprising water, microcapsules and starch particles is casted. Casting is not possible if the viscosity of the mixture is too high. Thus, during casting, the mixture of the invention has a dynamic viscosity of less than 60 Pa·s, preferably of less than 50 Pa·s and most preferably of less than 40 Pa·s.

In the context of the present invention, “dynamic viscosity” is measured as explained in paragraph [0093] of EP 2 410 865 B1.

The starch particles of the invention swell or dissolve in an aqueous mixture. Thus, dynamic viscosity of the mixture increases over time. In one embodiment of the invention, the method is done such that starch particles swell or dissolve only after casting such that the viscosity of the mixture comprising water, microcapsules and starch particles increases without cooling and without heating by at least 100% within 4 hours after casting. In a preferred embodiment of the invention, the viscosity of said mixture increases without cooling by at least 200% within 2 hours after casting.

Suitable starch particles that do not swell immediately and/or are not dissolved immediately. Preferably, starch particles as described in paragraph [0011] to paragraph [0014] of EP 2 410 865 B1 are used. Thus, the starch particles as used in the present invention have preferably an average particle size >1 μm and <500 μm.

The starch particles as used in the context of the present invention can be obtained by dissolution, gelatinization or plastification of at least one starch, optionally in combination with spray drying, roller drying or extrusion, and optionally milling.

Starch particles suitable for the present invention comprise preferably starch as described in paragraph [0015] to paragraph [0018] of EP 2 410 865 B1. Preferred starches are available inter alia at Cerestar (e.g. CreamTex 75725) and Emsland (e.g. Emden KH 15).

The mixture to be casted in accordance with the present comprises preferably at least 5 weight-% of at least one starch having a degree of polymerization in excess of 300, preferably in excess of 400 and most preferably in excess of 500. Such starch is known as long chain starch, as explained in paragraph [0015] of EP 2 410 865 EP B1. In this context, weight-% refers to the total weight of the mixture to be casted.

The casted multiparticulate solid dosage form of the invention has an elastic texture. Thus, the claimed multiparticulate solid dosage form is able to resume its shape spontaneously after being stretched or compressed. The person skilled in the art understands that the force which is used to stretch or compress the multiparticulate dosage form of the invention must be reasonable when doing such test. By way of example, the force must be lower than the force which is needed to destroy the dosage form. In a preferred embodiment, the texture of multiparticulate solid dosage form resembles the texture of gummy candies such as gummy bears. Thus, the multiparticulate solid dosage resumes its shape spontaneously after being stretched or compressed with two fingers.

Microcapsules according to the invention have typically an average diameter of from 1 μm to about 2000 μm. Due to this small size, hundreds of microcapsules may be embedded in the edible matrix. The casted multiparticulate solid dosage form according to the invention comprises preferably at least 100 microcapsules, more preferably at least 200 microcapsules, and most preferably more than 300 microcapsules.

Thus, a preferred embodiment of the present invention relates to a casted multiparticulate solid dosage form which has an elastic texture and which comprises at least 100 microcapsules, characterized in that said microcapsules have or do not have a functional coating.

The core (2a) of microcapsule (2) comprises at least one active ingredient. Depending on the nature of the active ingredient, the core may be liquid, solid or mixtures thereof. In one embodiment of the invention, the microcapsules encapsulate at least one hydrophobic or hydrophilic compound. Said compound may be a pharmaceutical drug and/or a micronutrient.

Examples of pharmaceutical drugs are opioids including μ-opioid receptor agonists such as alfentanil, buprenorphine, codeine, fentanyl, hydrocodone, hydromorphone, levomethadone, methadone, morphine, nalbuphine oxycodone, oxymorphone, pethidine, piritramid, remifentanil, sufentanil, tapentadol, tilidin, tramadol, and pharmaceutically acceptable salts thereof.

Thus, one embodiment of the present invention relates a casted multiparticulate solid dosage form which has an elastic texture and which comprises microcapsules,
characterized in that said microcapsules encapsulate a pharmaceutical drug such as an opioid, said opioid preferably selected from the group consisting of alfentanil, buprenorphine, codeine, fentanyl, hydrocodone, hydromorphone, levomethadone, methadone, morphine, nalbuphine oxycodone, oxymorphone, pethidine, piritramid, remifentanil, sufentanil, tapentadol, tilidin, tramadol, and pharmaceutically acceptable salts thereof.

Examples of micronutrients are vitamins, minerals, plant extracts, or oils such as microbial or marine oils.

Oil produced by a microorganism or obtained from a microbial cell is referred to as a “microbial oil”. Oil produced by algae and/or fungi is referred to as an algal and/or a fungal oil, respectively.

As used herein, a “microorganism” refers to organisms such as algae, bacteria, fungi, protist, yeast, and combinations thereof, e.g., unicellular organisms. A microorganism includes but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect. schmuckeri, and Pythium, including but not limited to Pythium insidiosum.

In one embodiment, the microorganisms are from the genus Mortierella, genus Crypthecodinium, genus Thraustochytrium, and mixtures thereof. In a further embodiment, the microorganisms are from Crypthecodinium Cohnii. In a further embodiment, the microorganisms are from Mortierella alpina. In a still further embodiment, the microorganisms are from Schizochytrium sp. In yet an even further embodiment, the microorganisms are selected from Crypthecodinium Cohnii, Mortierella alpina, Schizochytrium sp., and mixtures thereof.

In a still further embodiment, the microorganisms include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia.

In an even further embodiment, the microorganisms are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseurn, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. Species described within Ulkenia will be considered to be members of the genus Schizochytrium. In another embodiment, the microorganisms are from the order Thraustochytriales. In yet another embodiment, the microorganisms are from Thraustochytrium. In still a further embodiment, the microorganisms are from Schizochytrium sp.

In certain embodiments, the oil can comprise a marine oil. Examples of suitable marine oils include, but are not limited to, Atlantic fish oil, Pacific fish oil, or Mediterranean fish oil, or any mixture or combination thereof. In more specific examples, a suitable fish oil can be, but is not limited to, pollack oil, bonito oil, pilchard oil, tilapia oil, tuna oil, sea bass oil, halibut oil, spearfish oil, barracuda oil, cod oil, menhaden oil, sardine oil, anchovy oil, capelin oil, herring oil, mackerel oil, salmonid oil, tuna oil, and shark oil, including any mixture or combination thereof. Other marine oils suitable for use herein include, but are not limited to, squid oil, cuttle fish oil, octopus oil, krill oil, seal oil, whale oil, and the like, including any mixture or combination thereof.

Marine oil comprises omega fatty acids such as omega-6 fatty acids and/or omega-3 fatty acids. Manufacturing a multiparticulate solid dosage form that comprises omega-3 fatty acids such as docosahexaenoic acid (DHA) is particularly challenging because of the fishy off-flavor which occurs upon oxidation of the fatty acid.

One embodiment of the present invention relates to a casted multiparticulate solid dosage form which has an elastic texture and which comprises at least 200 microcapsules,
characterized in that said microcapsules do not have a functional coating, and/or
characterized in that said microcapsules encapsulate at least one micronutrient such as a vitamin, mineral, plant extract, oil or mixtures thereof.

When manufacturing the multiparticulate solid dosage of the invention, an aqueous mixture is casted. Whereas dispersing or dissolving hydrophobic liquids in an aqueous mixture is often not possible, it is possible to disperse microcapsules in an aqueous mixture because the hydrophobic liquid is surrounded by shell (2b). Good results are achieved when shell (2b) comprises or consists of a hydrocolloid such as modified starch, gelatin, polyphosphate, gum arabic, alginate, chitosan, carrageenan, pectin, carboxymethylcellulose or mixtures thereof.

Thus, a preferred embodiment of the present invention relates to a casted multiparticulate solid dosage form which has an elastic texture and which comprises at least 100 microcapsules, characterized in that each of said microcapsules encapsulates a hydrophobic liquid which comprises preferably at least one polyunsaturated fatty acid, and/or characterized in that each of said microcapsules have at least one shell, said shell preferably comprising or consisting of at least one hydrocolloid such as a modified starch, gelatin, polyphosphate, gum arabic, alginate, chitosan, carrageenan, pectin, carboxymethylcellulose or mixtures thereof.

In a preferred embodiment, the polyunsaturated fatty acid is in the form of a free fatty acid, salt, fatty acid ester (e.g., methyl or ethyl ester), monoacylglycerol (MAG), diacyglycerol (DAG) triacylglycerol (TAG), and/or phospholipid (PL) or mixtures thereof.

In an also preferred embodiment, the polyunsaturated fatty acid is an omega-3 fatty acid, an omega-6 fatty acid, or mixtures thereof. In the most preferred embodiment, the polyunsaturated fatty acid is docosahexaenoic acid (DHA).

Thus, a preferred embodiment of the present invention relates to a casted multiparticulate solid dosage form which has an elastic texture and which comprises at least 100 microcapsules, characterized in that said microcapsules encapsulates at least one polyunsaturated fatty acid, preferably selected from the group consisting of omega-3 fatty acid and omega-6 fatty acid (wherein docosahexaenoic acid is particularly preferred), and/or characterized in that said microcapsules have at least one shell, said shell preferably comprising or consisting of at least one hydrocolloid such as a modified starch, gelatin, polyphosphate, gum arabic, alginate, chitosan, carrageenan, pectin, carboxymethylcellulose or mixtures thereof.

In a particularly preferred embodiment of the invention, the casted multiparticulate solid dosage form comprises microcapsules as disclosed in WO 03/086104. The content of WO 03/086104 is hereby incorporated by reference. In said embodiment, the multiparticulate solid dosage form comprises agglomerations of primary microcapsules, each individual primary microcapsule having a primary shell and the agglomeration being encapsulated by an outer shell. Preferably, the primary shell and/or the outer shell comprise gelatin, polyphosphate, gum arabic, alginate, chitosan, carrageenan, pectin, carboxymethylcellulose or mixtures thereof. Said agglomerations are particularly suitable for casting.

Thus, in a preferred embodiment of the invention, the casted multiparticulate solid dosage form has an elastic texture,
wherein said multiparticulate solid dosage form comprises agglomerations of primary microcapsules, each individual primary microcapsule having a primary shell and the agglomeration being encapsulated by an outer shell, and
wherein the primary shell and/or the outer shell comprise gelatin, polyphosphate, gum arabic, alginate, chitosan, carrageenan, pectin, carboxymethylcellulose or mixtures thereof, and
wherein said primary shell encapsulates a hydrophobic liquid comprising preferably a polyunsaturated fatty acid.

In said embodiment, the polyunsaturated fatty acid is preferably in the form of a free fatty acid, salt, fatty acid ester (e.g., methyl or ethyl ester), monoacylglycerol (MAG), diacyglycerol (DAG) triacylglycerol (TAG), and/or phospholipid (PL) or mixtures thereof, wherein said polyunsaturated fatty acid is preferably an omega-3 fatty acid, an omega-6 fatty acid, or mixtures thereof.

Another embodiment of the invention relates to a casted multiparticulate solid dosage form which has an elastic texture and which comprises at least 100 microcapsules, characterized in that said microcapsules are microcapsules according to claim 1 of EP 1 736 060 B1 or according to claim 1 of EP 1 736 060 B2 or according to claim 1 of EP1492417 B1 or according to claim 1 of EP1492417 B2. All of these granted European Patents are member of the patent family of WO 03/086104 (vide supra). Thus, the content of EP 1 736 060131, EP 1 736 060 B2, EP1492417 B1 and EP1492417 B2 is hereby incorporated by reference, too.

To enhance patient compliance, the mixture to be casted is preferably fruit-flavored and/or contains sugar. In a preferred embodiment of the invention, sugar is replaced by at least one sweetener. Preferred sweeteners are maltitol syrup and steviol glycosides. Surprisingly, the elastic texture of the casted multiparticulate solid dosage form of the invention is not negatively affected if the multiparticulate solid dosage form comprises maltitol syrup and/or steviol glycosides as sweetener.

Thus, a preferred embodiment of the present invention relates to a casted multiparticulate solid dosage form which has an elastic texture and which comprises microcapsules and at least one sweetener,
characterized in that said microcapsules have a core (2a) and shell (2b), and
characterized in that said shell (2b) comprises at least one hydrocolloid such as modified starch, gelatin, polyphosphate, gum arabic, alginate, chitosan, carrageenan, pectin, carboxymethylcellulose or mixtures thereof, and
characterized in that said shell (2b) encapsulates a vitamin, a mineral, a plant extract, an oil or mixtures thereof and preferably encapsulates a hydrophobic liquid comprising preferably at least one polyunsaturated fatty acid such as docosahexaenoic acid (DHA).

The casted multiparticulate solid dosage form of the invention has preferably a total weight from 1 g to 6 g, preferably from 2 g to 5 g and most preferably from 3 g to 4 g. In preferred embodiment of the invention, the casted multiparticulate solid dosage form comprises at least 10 mg, preferably at least 20 mg and most preferably at least 30 mg of at least one polyunsaturated fatty acid.

Thus, an even more preferred embodiment of the present invention relates to relates to a casted multiparticulate solid dosage form which has an elastic texture and which comprises microcapsules,
characterized in that a multiparticulate solid dosage form has a weight from 1 g to 6 g, and
characterized in that said microcapsules have a core (2a) and shell (2b), and
characterized in that said shell (2b) comprises at least one hydrocolloid such as modified starch, gelatin, polyphosphate, gum arabic, alginate, chitosan, carrageenan, pectin, carboxymethylcellulose or mixtures thereof, and
characterized in that said shell (2b) encapsulates a hydrophobic liquid comprising an omega-3 fatty acid, an omega-6 fatty acid and/or mixtures thereof, and
characterized in that said multiparticulate solid dosage form comprises at least 10 mg, preferably at least 20 mg and most preferably at least 30 mg of at least one polyunsaturated fatty acid.

In said even more preferred embodiment, the omega-3 fatty acid and/or the omega-6 fatty acid is preferably in the form of a free fatty acid, a salt, a fatty acid ester (e.g., methyl or ethyl ester), a monoacylglycerol (MAG), a diacyglycerol (DAG), a triacylglycerol (TAG), a phospholipid (PL) or mixtures thereof.

Multiparticulate solid dosage form according to the invention is obtainable by the method of the present invention.

The present invention relates to a method of producing a multiparticulate solid dosage form which has an elastic texture,
wherein a mixture comprising water, microcapsules and starch particles is casted, and
wherein said mixture comprises at least 5 weight-% of at least one starch having preferably a degree of polymerization in excess of 300, based on the total weight of the mixture, and
characterized in that said microcapsules encapsulate at least one pharmaceutical drug and/or at least one micronutrient.

The mixture to be used in the method of the invention must be suitable for casting, i.e. the mixture's viscosity must be reasonably low. In a preferred embodiment of the invention, said mixture has during casting a dynamic viscosity of less than 60 Pa·s, preferably of less than 50 Pa·s and most preferably of less than 40 Pa·s.

The starch particles of the mixture swell or dissolve only after casting such that the viscosity of said mixture increases without cooling or heating after casting. In a preferred embodiment of the invention, the viscosity of said mixture increases without cooling and without heating by at least 100% when comparing the viscosity of said mixture 2 hours after its preparation with the viscosity of the same mixture 6 hours after its preparation.

Viscosity of the mixture is influenced by various factors such as the amount of starch and the starch's degree of polymerization. The person skilled in the art knows how to determine the degree of polymerization (DP). In one embodiment of the invention, the degree of polymerization (DP) is determined as described in “Determination of the degree of polymerization of oligosaccharides” by Stewart, L.; Nordin, P. Analytical Biochemistry, February 1963, 5(2):175-178.

Preferably, the mixture comprises from 10 weight-% to 40 weight-% particulate starch, preferably from 15 weight-% to 35 weight-% particulate starch and most preferably from 20 weight-% to 25 weight-% particulate starch, based on the total weight of the mixture.

Thus, a preferred embodiment of the invention relates to a method of producing a multiparticulate solid dosage form which has an elastic texture,
wherein a mixture comprising water, microcapsules and starch particles is casted, and
wherein said microcapsules encapsulate at least one pharmaceutical drug and/or at least one micronutrient,
characterized in that the viscosity of said mixture increases without cooling and without heating by at least 100% when comparing the viscosity of said mixture 2 hours after its preparation with the viscosity of the same mixture 6 hours after its preparation.

An also preferred embodiment of the invention relates to a method of producing a multiparticulate solid dosage form which has an elastic texture,
wherein a mixture comprising water, microcapsules and particulate starch is casted, and
wherein said mixture comprises 10 weight-% to 40 weight-% particulate starch, based on the total weight of the mixture starch particles, and
wherein said microcapsules encapsulate at least one pharmaceutical drug and/or at least one micronutrient,
characterized in that said mixture comprises at least 5 weight-% of at least one starch having a degree of polymerization in excess of 300, based on the total weight of the mixture.

In these preferred embodiments, the microcapsules as herein before described are preferably used.

For casting, any suitable mold can be used. The shape of the obtained multiparticulate solid dosage form is obtained by the shape of the chosen mold. The casted multiparticulate solid dosage form has preferably not the shape of a tablet. Preferred shapes are cone, sphere, cylinder or pyramid, wherein a casted multiparticulate solid dosage form is particularly preferred.

The casted multiparticulate solid dosage form comprises preferably less than 10 weight-%, more preferably less than 5 weight-% and most preferably less than 2 weight-% water, based on the total weight of the multiparticulate solid dosage form. Thus, depending on the amount of water in the mixture which is casted, a drying step may be necessary after casting to remove at least some of the water.

Any suitable packaging material can be used to package the casted multiparticulate solid dosage forms according to the invention. In a preferred embodiment of the invention, the casted multiparticulate solid dosage forms are packaged in boxes, bottles, blisters or bags. Packaging is particularly space-saving if the casted multiparticulate solid dosage form of the invention has the shape of a cone.

Patient compliance is poor when an active has an off-flavor. This applies in particular to actives which have a fishy off-flavor. Surprisingly, patient compliance can be enhanced when the casted multiparticulate solid dosage of the invention is used. Patients appear to appreciate the taste, shape and/or texture of the casted multiparticulate solid dosage forms according to the invention.

Thus, the present invention also relates to the use of the casted multiparticulate solid dosage form of the invention for enhancing patient compliance with active ingredients which have preferably an off-flavor such as a fishy off-flavor. In a preferred embodiment, the casted multiparticulate solid dosage form for said use is fruit-flavored.

### EXAMPLES

The present invention is further illustrated by the following examples.

### Example 1

In example 1, docosahexaenoic acid (DHA) has been chosen as model substance. DHA is an omega-3 fatty acid. Like most other omega-3 fatty acids, DHA is prone to oxidation. Upon oxidation, DHA gets a fishy off-flavor that is easily recognized upon consumption, even at very low quantities.

In example 1, MEG-3® DHA H Powder (available from DSM®) is mixed with water, starch particles, sugar, fruit-flavour and other auxiliary compounds. The obtained mixture had a dynamic viscosity of less than 60 Pa·s, i.e. was liquid enough to be casted as explained in example 1 of EP 2 015 642 B1.

Multiparticulate solid dosage forms in the shape of cones where thus obtained. The cones had an elastic texture, similar to gummy candies. Each cone had a total weight of 3.6 g and comprised 40 mg DHA.

No fishy taste has been recognized by the sensory panel.

### Example 2

Example 1 is repeated. However, in example 2, sugar is replaced by sweeteners (maltitol syrup and steviol glycosides). Cones having an elastic texture were casted, similar to example 1.

A direct comparison of the cones of example 1 (with sugar) with the cones of example 2 (sugar replaced by sweeteners) revealed a minor difference in terms of sweetness. However, neither the cones of example 1 nor the cones of example 2 had a fishy off-flavor.

Elasticity of the cones of example 2 were similar to the elasticity of the cones of example 1.

### Example 3

Example 1 is repeated. However, in example 3, MEG-3® DHA H Powder was replaced by Life's DHA® S24-P100 powder (also available from DSM®). Life's DHA® S24-P100 comprises microcapsules that encapsulate DHA from a vegetarian source.

Cones with an elastic texture were manufactured, similar to example 1. No fishy taste has been recognized by the sensory panel.

1. Method of producing a multiparticulate solid dosage form which has an elastic texture,
wherein a mixture comprising water, microcapsules and starch particles is casted, and
wherein said mixture comprises at least 5 weight-% of at least one starch having a degree of polymerization in excess of 300, based on the total weight of the mixture, and
characterized in that said microcapsules encapsulate at least one pharmaceutical drug and/or at least one micronutrient.

2. Method according to claim 1, wherein said mixture has during casting a dynamic viscosity of less than 60 Pa·s, and/or wherein the viscosity of said mixture increases without cooling and without heating by at least 100% when comparing the viscosity of said mixture 2 hours after its preparation with the viscosity of the same mixture 6 hours after its preparation.

3. Method according to claim 1, wherein said mixture comprises from 10 weight-% to 40 weight-% particulate starch, preferably from 15 weight-% to 35 weight-% particulate starch and most preferably from 20 weight-% to 25 weight-% particulate starch, based on the total weight of the mixture.

4. Method according to claim 1, wherein said mixture comprises agglomerations of primary microcapsules, each individual primary microcapsule having a primary shell and the agglomerations being encapsulated by an outer shell.

5. Method according to claim 4, wherein the primary shell and/or the outer shell comprise gelatin, polyphosphate, gum arabic, alginate, chitosan, carrageenan, pectin, carboxymethylcellulose or mixtures thereof.

6. Method according to claim 1, wherein said microcapsules encapsulate a hydrophobic liquid, said liquid preferably comprising at least one polyunsaturated fatty acid.

7. Method according to claim 1, wherein the micronutrient is a polyunsaturated fatty acid and wherein the polyunsaturated fatty acid is in the form of a free fatty acid, a salt, an ester, a monoacylglycerol (MAG), a diacylglycerol (DAG), a triacylglycerol (TAG), a phospholipid (PL) or mixtures thereof.

8. Method according to claim 6, wherein the polyunsaturated fatty acid is an omega-3 fatty acid, an ester of an omega-3 fatty acid, an omega-6 fatty acid, an ester of an omega-6 fatty acid or mixtures thereof.

9. Casted multiparticulate solid dosage form comprising at least 100 microcapsules, wherein said casted multiparticulate solid dosage form has a weight from 1 g to 6 g, and/or wherein said casted multiparticulate solid dosage form comprises at least 10 mg of at least one polyunsaturated fatty acid.

10. Casted multiparticulate solid dosage form according to claim 9, wherein said casted multiparticulate solid dosage form is obtained wherein said casted multiparticulate solid dosage form comprises preferably less than 5 weight-% water, based on the total weight of the multiparticulate solid dosage form.

11. Casted multiparticulate solid dosage form according to claim 9, wherein said casted multiparticulate solid dosage form has weight from 2 g to 5 g, preferably from 3 g to 4 g and/or wherein said casted multiparticulate solid dosage form comprises at least 20 mg, preferably at least 30 mg of at least one polyunsaturated fatty acid.

12. Casted multiparticulate solid dosage form according to claim 9, wherein said casted multiparticulate solid dosage form has not the shape of a tablet and has preferably the shape of cone, sphere, cylinder or pyramid.

13. Use of a mogul line for casting a multiparticulate solid dosage form, wherein said multiparticulate solid dosage form comprises at least 100 microcapsules.

14. Use according to claim 13, wherein said multiparticulate solid dosage form is the casted multiparticulate solid dosage form.

15. Package comprising at least one multiparticulate solid dosage form according to claim 9, wherein said multiparticulate solid dosage form has the shape of a cone.
